(19) United States
(12) Patent Application Publication
(10) Pub. No.: US 2023/0097458 A1
(43) Pub. Date: Mar. 30, 2023

(54) AIR PURIFIER WITH PHOTOCATALYST

(71) Applicant: Airplove (Xiamen) Electronic Co., Ltd., Xiamen (CN)

(72) Inventors: Jueyuan Chen, Xiamen (CN); Huiping Cai, Xiamen (CN); Longzhe Chen, Xiamen (CN)

(21) Appl. No.: 17/653,907

(22) Filed: Mar. 8, 2022

(30) Foreign Application Priority Data
Sep. 29, 2021 (CN) 202122380013.4

(51) Int. Cl. B01D 53/88; B01D 53/00; B01D 46/24; A61L 9/20

(52) U.S. Cl. CPC B01D 53/885; B01D 53/007; B01D 46/24; A61L 9/205; B01D 2259/804; B01D 2255/802; A61L 2209/12

(57) ABSTRACT

The present invention discloses an air purifier with a photocatalyst, including an air intake structure and an air discharge structure. The air intake structure is provided with a purification cavity, and a connection seat is provided in the purification cavity and is provided with a photocatalyst purification member detachably mounted to the connection seat. The air purifier with a photocatalyst of the present invention has a simple structure and a rational design, and demounting and mounting operations of photocatalyst purification member are simple, easy and quick, thereby effectively improving the demounting and mounting efficiency of the photocatalyst purification member.

### TECHNICAL FIELD

The present invention relates to the field of air purifiers, and in particular to an air purifier with a photocatalyst.

### BACKGROUND

Photocatalyst purification is a common air purification method in air purifiers at present. An air purifier with a photocatalyst directly decomposes or kills toxic and harmful substances such as toxic gases, bacteria, molds and viruses by means of a chemical method, which is more thorough and effective than physical adsorption to purify air.

The existing air purification apparatus with a photocatalyst directly fixes the photocatalyst and its carrier in the apparatus. The photocatalyst and its carrier themselves have a certain service life and need to be cleaned or replaced regularly. However, the way of fixing the photocatalyst and its carrier directly in the apparatus brings inconvenience to the cleaning or disassembling and replacement.

In view of this, the inventor of the present application has invented an air purifier with a photocatalyst.

### SUMMARY

An objective of the present invention is to provide an air purifier with a photocatalyst, which can realize simple demounting and mounting of a photocatalyst component.

To achieve the above objective, the present invention adopts the following technical scheme: an air purifier with a photocatalyst, including: an air intake structure and an air discharge structure, where the air intake structure is provided with a purification cavity, and a connection seat is provided in the purification cavity and is provided with a photocatalyst purification member detachably mounted to the connection seat.

Further, the connection seat is columnar, the photocatalyst purification member has a mounting through hole, and the photocatalyst purification member is mounted and sleeved around the connection seat by means of the mounting through hole.

Further, an out wall of the connection seat is provided with a positioning protrusion, and an inner wall of the mounting through hole is provided with a positioning groove adapted to the positioning protrusion.

Further, the positioning protrusion is arranged in an axial direction of the connection seat and is in the shape of an elongated strip, the positioning groove is correspondingly in the shape of an elongated strip, and two ends of the positioning groove are not closed so that a through groove is formed.

Further, the photocatalyst purification member includes a housing, a UV lamp provided in the housing, and a photocatalyst plate.

Further, there are three photocatalyst plates, which are uniformly arranged in a circumferential direction of the housing, and the UV lamp is arranged inside the housing.

Further, the air intake structure is located below the air discharge structure, and the air intake structure is connected and fixed to the air discharge structure via a quick-release structure; a clearance hole in communication with the purification cavity is provided at a top end of the air intake structure; and the connection seat is perpendicularly arranged at the center of an inner bottom wall of the purification cavity.

Further, a metal probe is provided at atop end of the photocatalyst purification member, and a PCB contact, which is arranged corresponding to the probe, is provided at a bottom end of the air discharge structure.

Further, a male coupler end is provided at a top end of the connection seat; and a female coupler end, which is adapted and connected to the male coupler end, is provided at the bottom end of the air discharge structure.

Further, the air intake structure further includes an annular filter screen, which is placed into the purification cavity through the clearance hole, and the connection seat is located in the filter screen.

After adopting the technical scheme, the present invention has the following advantages:

The air purifier with a photocatalyst of the present invention has a simple structure and a rational design, and demounting and mounting operations of photocatalyst purification member are simple, easy and quick, thereby effectively improving the demounting and mounting efficiency of the photocatalyst purification member.

### BRIEF DESCRIPTION OF DRAWINGS

FIG. 1 is a schematic perspective view of an air purifier according to an embodiment of the present invention;

FIG. 2 is an exploded schematic view of the air purifier according to the embodiment of the present invention;

FIG. 3 is a schematic perspective view of an air intake structure according to the embodiment of the present invention;

FIG. 4 is an exploded schematic view of the air intake structure according to the embodiment of the present invention;

FIG. 5 is a schematic view of the air intake structure according to the embodiment of the present invention with a photocatalyst purification member and a filter screen hidden;

FIG. 6 is a schematic view of the photocatalyst purification member according to the embodiment of the present invention; and

FIG. 7 is a schematic view, from the bottom, of an air discharge structure according to the embodiment of the present invention.

### DESCRIPTION OF REFERENCE SIGNS

10—Air intake structure,
11—Purification cavity,
12—Connection seat, 121—Positioning protrusion, 122—Male coupler end,
13—Photocatalyst purification member, 131—Housing, 132—Photocatalyst plate, 133—Probe,
14—Mounting through hole, 141—Positioning groove,
15—Clearance hole,
16—Filter screen,
20—Air discharge structure,
21—PCB contact, and
22—Female coupler end.

### DETAILED DESCRIPTION

In order to make the objectives, technical schemes and advantages of the present invention more apparent, the present invention is further described in detail in conjunction with the accompanying drawings and embodiments. It should be understood that the specific embodiments described here are intended only to explain the present invention and are not intended to limit the present invention.

In the present invention, it should be noted that the terms “up”, “down”, “left”, “right”, “vertical”, “horizontal”, “inside” and “outside” are all based on the orientation or positional relationship shown in the drawings, only for the convenience of describing the present invention and simplifying the description, and do not indicate or imply that the device or element of the present invention must have a specific orientation, so it cannot be understood as limiting the present invention.

### Embodiment

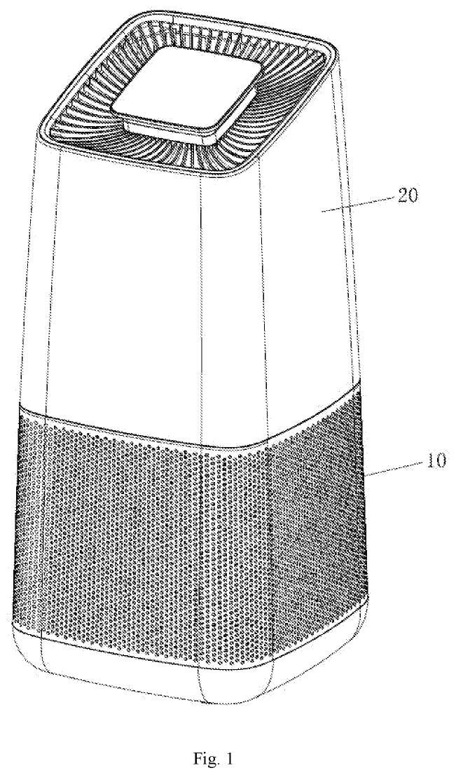

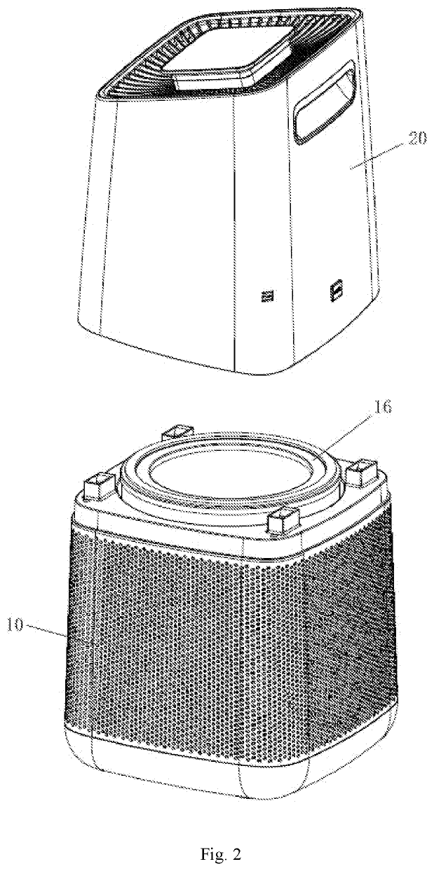

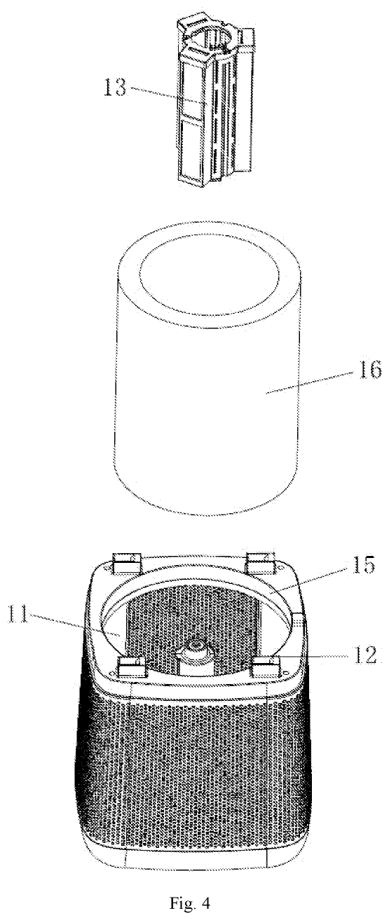

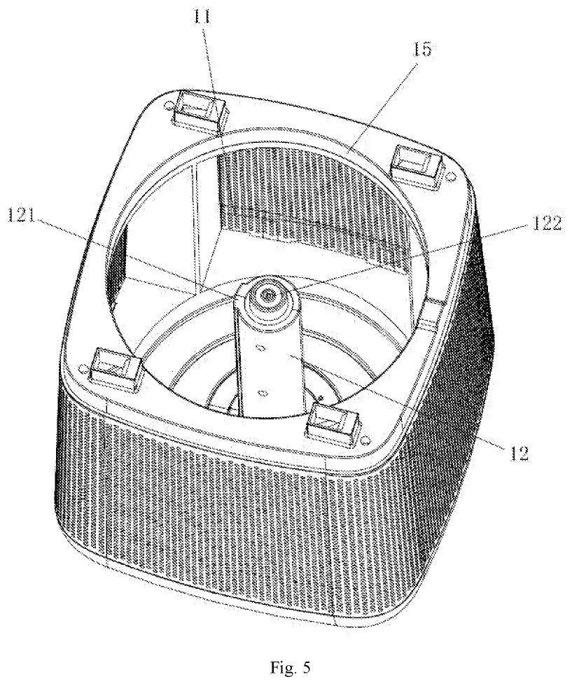

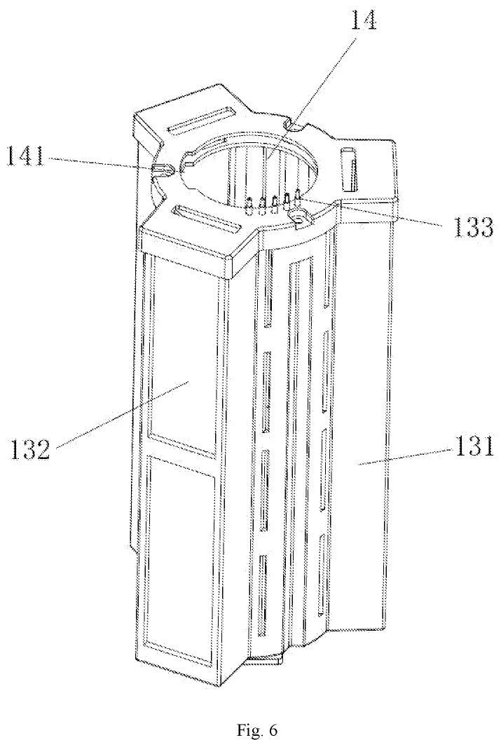

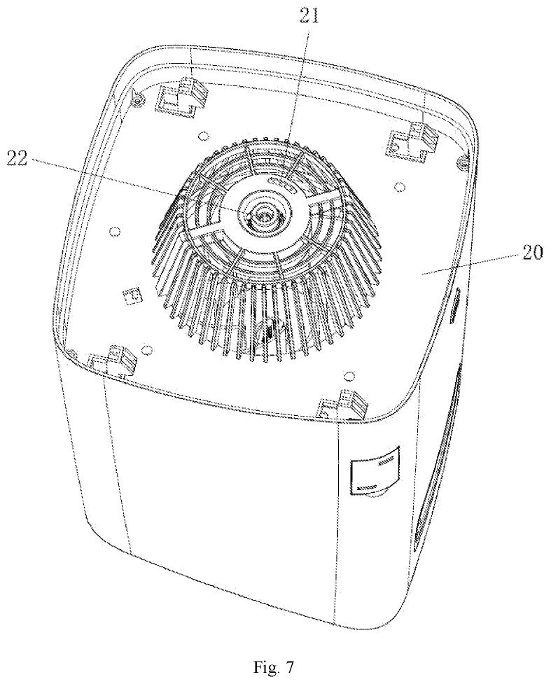

Referring to FIGS. 1 to 7, the present invention discloses an air purifier with a photocatalyst, including an air intake structure 10 and an air discharge structure 20. The air intake structure 10 is provided with a purification cavity 11, and a connection seat 12 is provided in the purification cavity 11, and the connection seat 12 is provided with a photocatalyst purification member 13 detachably mounted to the connection seat. The photocatalyst purification member 13 is of an integrated design. During replacement, the photocatalyst purification member 13 can be directly removed and replaced as a whole, which is easy and quick.

Specifically, the connection seat 12 is columnar, the photocatalyst purification member 13 has a mounting through hole 14 in the middle, and the photocatalyst purification member 13 is mounted and sleeved around the connection seat 12 by means of the mounting through hole 14. During mounting, the photocatalyst purification member 13 is directly snap-fitted into the connection seat 12, and during demounting, the photocatalyst purification member 13 is directly pulled out along the connection seat 12, which is easy and quick, and effectively improves the efficiency.

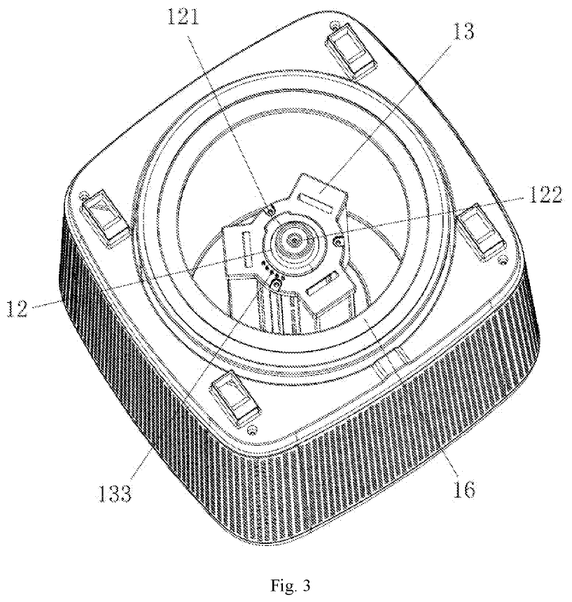

Referring to FIGS. 3 to 6, an out wall of the connection seat 12 is provided with a positioning protrusion 121, and an inner wall of the mounting through hole 14 is provided with a positioning groove 141 adapted to the positioning protrusion 121. More specifically, the positioning protrusion 121 is arranged in an axial direction of the connection seat 12 and is in the shape of an elongated strip, the positioning groove 141 is correspondingly in the shape of an elongated strip, and two ends of the positioning groove 141 are not closed, so that the positioning groove 141 becomes a through groove with two open ends. The design of the positioning protrusion 121 and the positioning groove 141 enables the photocatalyst purification member 13 to be mounted accurately and quickly, and can also effectively prevent the rotation of the photocatalyst purification member when in use, ensuring its stability.

In this embodiment, the photocatalyst purification member 13 includes: a housing 131, a UV lamp (not shown) provided in the housing 131, and a photocatalyst plate 132. More specifically, there are three photocatalyst plates 132, which are uniformly arranged in a circumferential direction of the housing 131 of the photocatalyst purification member 13, and the UV lamp, that is, an ultraviolet lamp tube, is located inside the housing 131.

Referring to FIGS. 1 to 7, more specifically, the air intake structure 10 is located below the air discharge structure 20, and the air intake structure 10 is connected and fixed to the air discharge structure 20 via a quick-release structure. A clearance hole 15 in communication with the purification cavity 11 is provided at a top end of the air intake structure 10, and the connection seat 12 is perpendicularly arranged at the center of an inner bottom wall of the purification cavity 11. During mounting of the photocatalyst purification member 13, the air intake structure 10 is separated from the air discharge structure 20 by means of the quick-release structure, the photocatalyst purification member 13 passes through the clearance hole 15 and is inserted, from the top to the bottom, from the top end of the connection seat 12 to the connection seat 12, and finally the air intake structure 10 and the air discharge structure 20 are assembled by means of the quick-release structure. The whole process is simple, easy and quick to operate. The quick-release structure may be any existing structure that can provide the corresponding function herein, which is not limited here.

A metal probe 133 is provided at a top end of the photocatalyst purification member 13, and a PCB contact 21, which is arranged corresponding to the probe 133, is provided at a bottom end of the air discharge structure 20. After the air purifier is assembled, the UV lamp is powered on through the corresponding contact conduction between the probe 133 and the PCB contact 21, which makes the energizing of the photocatalyst purification member simple and quick. In addition, a male coupler end 122 is provided at a top end of the connection seat 12; and a female coupler end 22, which is adapted and connected to the male coupler end 122, is provided at the bottom end of the air discharge structure 20. After the air purifier is assembled, the energizing of the air discharge structure 20 and the air intake structure 10 is enabled by means of the female coupler end 22 and the male coupler end 122, which is simple and quick.

The air intake structure 10 further includes an annular filter screen 16. The annular filter screen 16 is placed into the purification cavity 11 through the clearance hole 15, and the connection seat 12 is located in the filter screen 16. The outside air enters the purification cavity 11 of the air intake structure 10, and impurities such as dust particles are first filtered out by the filter screen 16. The air then enters the photocatalyst purification member 13 in the filter screen 16 for purification and sterilization, and finally enters the air discharge structure 20 above the air intake structure 10 and is discharged to the outside of the purifier. In this way, it is also possible to make the replacement of the filter screen 16 easy and quick.

Only a preferred embodiment of the present invention is described above, and the scope of protection of the present invention is not limited thereto. Any changes or substitutions that can be readily conceived by those skilled in the art within the technical scope disclosed by the present invention shall fall within the scope of protection of the present invention. Therefore, the scope of protection of the present invention should be subject to the scope of protection of the claims.

1. An air purifier with a photocatalyst, comprising: an air intake structure and an air discharge structure, wherein the air intake structure is provided with a purification cavity, and a connection seat is provided in the purification cavity and is provided with a photocatalyst purification member detachably mounted to the connection seat.

2. The air purifier with a photocatalyst of claim 1, wherein the connection seat is columnar, the photocatalyst purification member has a mounting through hole, and the photocatalyst purification member is mounted and sleeved around the connection seat by means of the mounting through hole.

3. The air purifier with a photocatalyst of claim 2, wherein an out wall of the connection seat is provided with a positioning protrusion, and an inner wall of the mounting through hole is provided with a positioning groove adapted to the positioning protrusion.

4. The air purifier with a photocatalyst of claim 3, wherein the positioning protrusion is arranged in an axial direction of the connection seat and is in the shape of an elongated strip, the positioning groove is correspondingly in the shape of an elongated strip, and two ends of the positioning groove are not closed so that a through groove is formed.

5. The air purifier with a photocatalyst of claim 1, wherein the photocatalyst purification member comprises a housing, a UV lamp provided in the housing, and a photocatalyst plate.

6. The air purifier with a photocatalyst of claim 5, wherein there are three photocatalyst plates, which are uniformly arranged in a circumferential direction of the housing, and the UV lamp is arranged inside the housing.

7. The air purifier with a photocatalyst of claim 1, wherein the air intake structure is located below the air discharge structure, and the air intake structure is connected and fixed to the air discharge structure via a quick-release structure; a clearance hole in communication with the purification cavity is provided at a top end of the air intake structure; and the connection seat is perpendicularly arranged at the center of an inner bottom wall of the purification cavity.

8. The air purifier with a photocatalyst of claim 7, wherein a metal probe is provided at a top end of the photocatalyst purification member, and a PCB contact, which is arranged corresponding to the probe, is provided at a bottom end of the air discharge structure.

9. The air purifier with a photocatalyst of claim 7, wherein a male coupler end is provided at a top end of the connection seat; and a female coupler end, which is adapted and connected to the male coupler end, is provided at the bottom end of the air discharge structure.

10. The air purifier with a photocatalyst of claim 7, wherein the air intake structure further comprises an annular filter screen, which is placed into the purification cavity through the clearance hole, and the connection seat is located in the filter screen.

11. The air purifier with a photocatalyst of claim 2, wherein the air intake structure is located below the air discharge structure, and the air intake structure is connected and fixed to the air discharge structure via a quick-release structure; a clearance hole in communication with the purification cavity is provided at a top end of the air intake structure; and the connection seat is perpendicularly arranged at the center of an inner bottom wall of the purification cavity.

12. The air purifier with a photocatalyst of claim 3, wherein the air intake structure is located below the air discharge structure, and the air intake structure is connected and fixed to the air discharge structure via a quick-release structure; a clearance hole in communication with the purification cavity is provided at a top end of the air intake structure; and the connection seat is perpendicularly arranged at the center of an inner bottom wall of the purification cavity.

13. The air purifier with a photocatalyst of claim 4, wherein the air intake structure is located below the air discharge structure, and the air intake structure is connected and fixed to the air discharge structure via a quick-release structure; a clearance hole in communication with the purification cavity is provided at a top end of the air intake structure; and the connection seat is perpendicularly arranged at the center of an inner bottom wall of the purification cavity.

14. The air purifier with a photocatalyst of claim 5, wherein the air intake structure is located below the air discharge structure, and the air intake structure is connected and fixed to the air discharge structure via a quick-release structure; a clearance hole in communication with the purification cavity is provided at a top end of the air intake structure; and the connection seat is perpendicularly arranged at the center of an inner bottom wall of the purification cavity.

15. The air purifier with a photocatalyst of claim 6, wherein the air intake structure is located below the air discharge structure, and the air intake structure is connected and fixed to the air discharge structure via a quick-release structure; a clearance hole in communication with the purification cavity is provided at a top end of the air intake structure; and the connection seat is perpendicularly arranged at the center of an inner bottom wall of the purification cavity.
